(19) United States
(12) Patent Application Publication
(10) Pub. No.: US 2017/0319079 A1
(43) Pub. Date: Nov. 9, 2017

(54) METHOD OF CALCULATING BLOOD PRESSURE USING A MOBILE DEVICE

(71) Applicant: SMN Medical P.C., Rye, NY (US)

(72) Inventors: Denis Mezenok, Kryvvi Rih (UA); Artem Kalmykov, Kharkiv (UA); Alexey Bondarchyk, Korosten (UA)

(21) Appl. No.: 15/149,863

(22) Filed: May 9, 2016

(51) Int. Cl. A61B 5/021; A61B 5/00; A61B 5/026; A61B 5/024; A61B 7/04

(52) U.S. Cl. CPC A61B 5/021; A61B 7/04; A61B 5/7278; A61B 5/6898; A61B 5/0022; A61B 5/6824; A61B 5/026; A61B 5/02438; A61B 5/6826

(57) ABSTRACT

Apparatus and associated methods relate to determining a user's blood pressure via information collected by a camera and a microphone of a portable communication device (PCD). In an illustrative example, the PCD simultaneously records a blood flow and a pulsation for the user. The blood flow may be recorded by positioning the camera such that camera aligns with a finger of a first arm of the user. The pulsation may be recorded by positioning the microphone such that the microphone proximate to an antecubital artery of the second arm of the user. In response, the PCD uses the recorded blood flow and pulsation information to determine a systolic blood pressure and diastolic blood pressure for the user in accordance with previously input body parameters (e.g., height, weight, age, gender) of the user.

### TECHNICAL FIELD

Various embodiments relate generally to blood pressure measurement devices.

### BACKGROUND

In the medical field, it is common practice to monitor the vital signs of a patient. Vital signs typically include a body temperature, a blood pressure, a pulse, and a breathing rate of a patient. Doctors commonly use vital signs when administering a diagnosis of a patient or determining a treatment plan for the patient.

Various medical equipment may be used to measure a patient's vital signs. For example, a digital thermometer may be used to measure a patient's body temperature. A stethoscope and blood pressure cuff may be used to measure the blood pressure of a patient. While some types of medical equipment may be found in a patient's home such as a thermometer, for example, the medical equipment necessary to monitor vitals requires a visit to a doctor's office, a medical facility, or a medical transportation vehicle (e.g., ambulance).

### SUMMARY

Apparatus and associated methods relate to determining a user's blood pressure via information collected by a camera and a microphone of a portable communication device (PCD). In an illustrative example, the PCD simultaneously records a blood flow and a pulsation for the user. The blood flow may be recorded by positioning the camera such that camera aligns with a finger of a first arm of the user. The pulsation may be recorded by positioning the microphone such that the microphone proximate to an antecubital artery of the second arm of the user. In response, the PCD uses the recorded blood flow and pulsation information to determine a systolic blood pressure and diastolic blood pressure for the user in accordance with previously input body parameters (e.g., height, weight, age, gender) of the user.

Various embodiments may achieve one or more advantages. For example, some embodiments may permit the user to transfer the user's inputted body parameters and determined blood pressure to a medical facility, or a doctor' office, for medical diagnosis. The user's inputted body parameters and determined blood pressure may be stored in a data memory to create a metrics history for the user. The stored information may be associated with a unique user profile for the user. The unique user profile, in accordance with the stored information, may provide individual trends and patterns for the user. The individual trends and patterns may assist in a medical diagnosis administered by a healthcare practitioner or self-care by the patient.

In some embodiments, the unique user profile may increase accuracy for determining blood pressure by tailoring an algorithm for specific use with the unique user profile. The algorithm may be updated in real-time to reflect the most recent stored information for the user associated with the unique user profile.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary use of a PCD for measuring a blood pressure of a user.

FIG. 2 depicts a schematic view of an exemplary PCD for measuring a blood pressure of a user.

FIG. 3 depicts a home screen for an exemplary blood pressure measuring application running on a PCD.

FIG. 4 depicts a flowchart for an exemplary method of measuring a user's blood pressure with a PCD.

FIG. 5 depicts a flowchart of an exemplary method of using a PCD to capture image frames and sound samples.

FIG. 6 a flowchart of an exemplary method of using a PCD to measure a blood pressure for a user.

FIG. 7 depicts an illustration of an exemplary blood pressure monitoring management system (BPMMS) integrated for use multi-use applications.

FIG. 8 depicts a flowchart of an exemplary PCD-BPMMS operations executed between a PCD and a BPMMS.

Like reference symbols in the various drawings indicate like elements.

### DETAILED DESCRIPTION OF ILLUSTRATIVE EMBODIMENTS

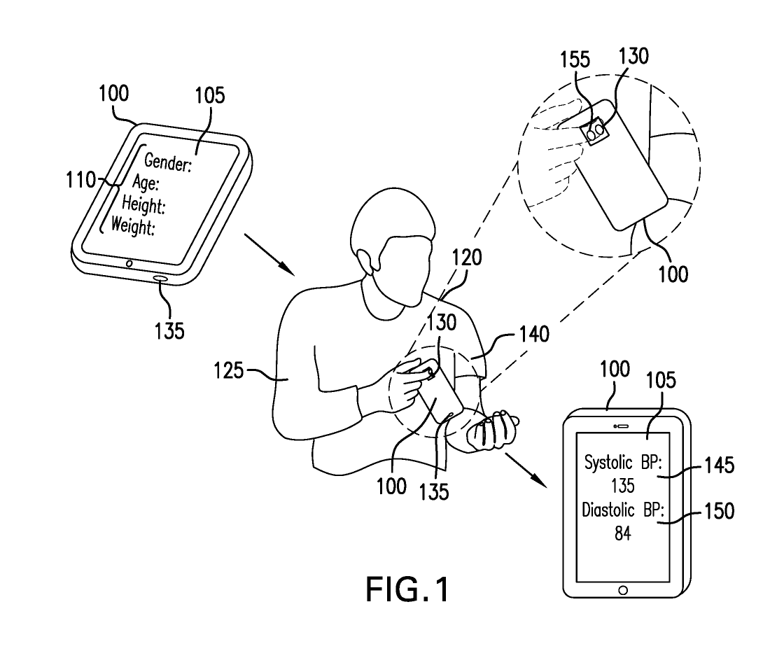

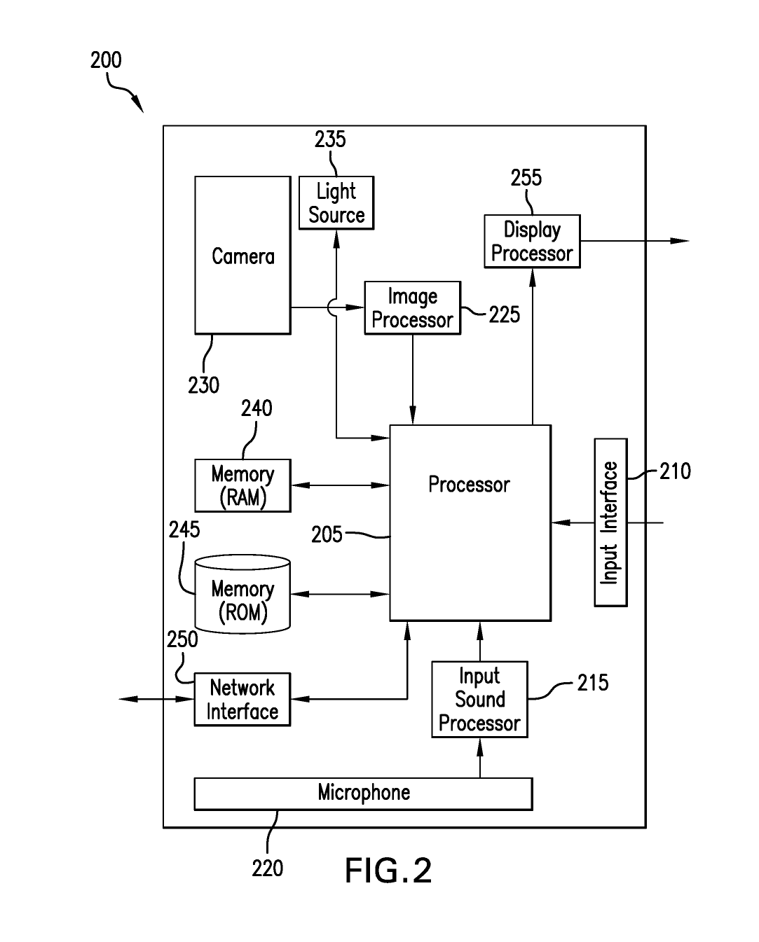

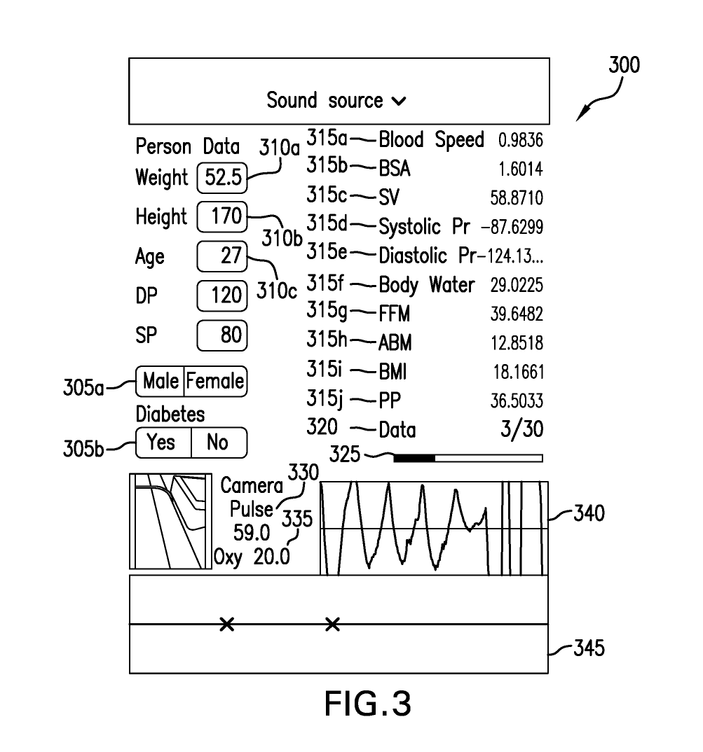

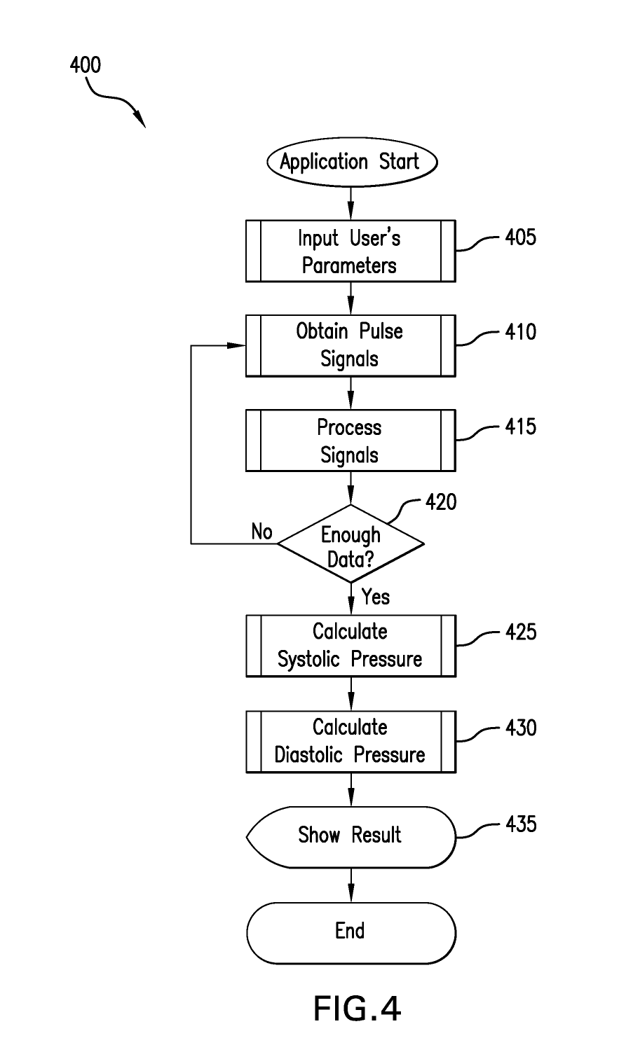

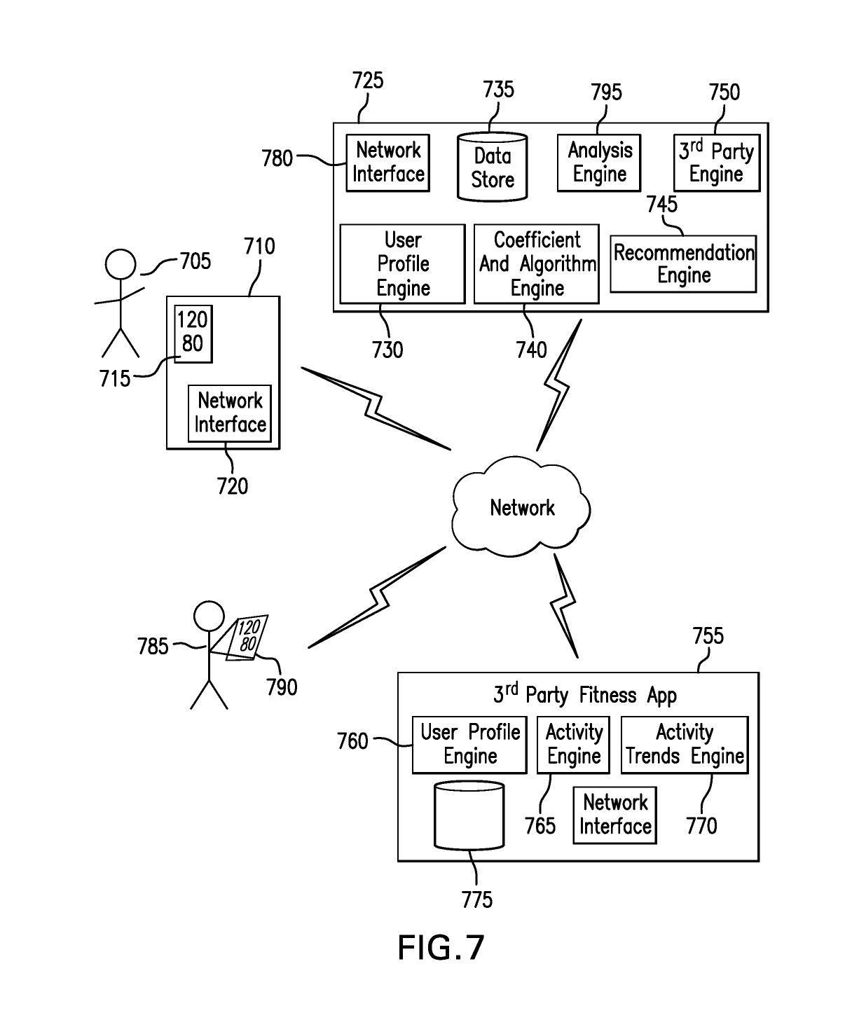

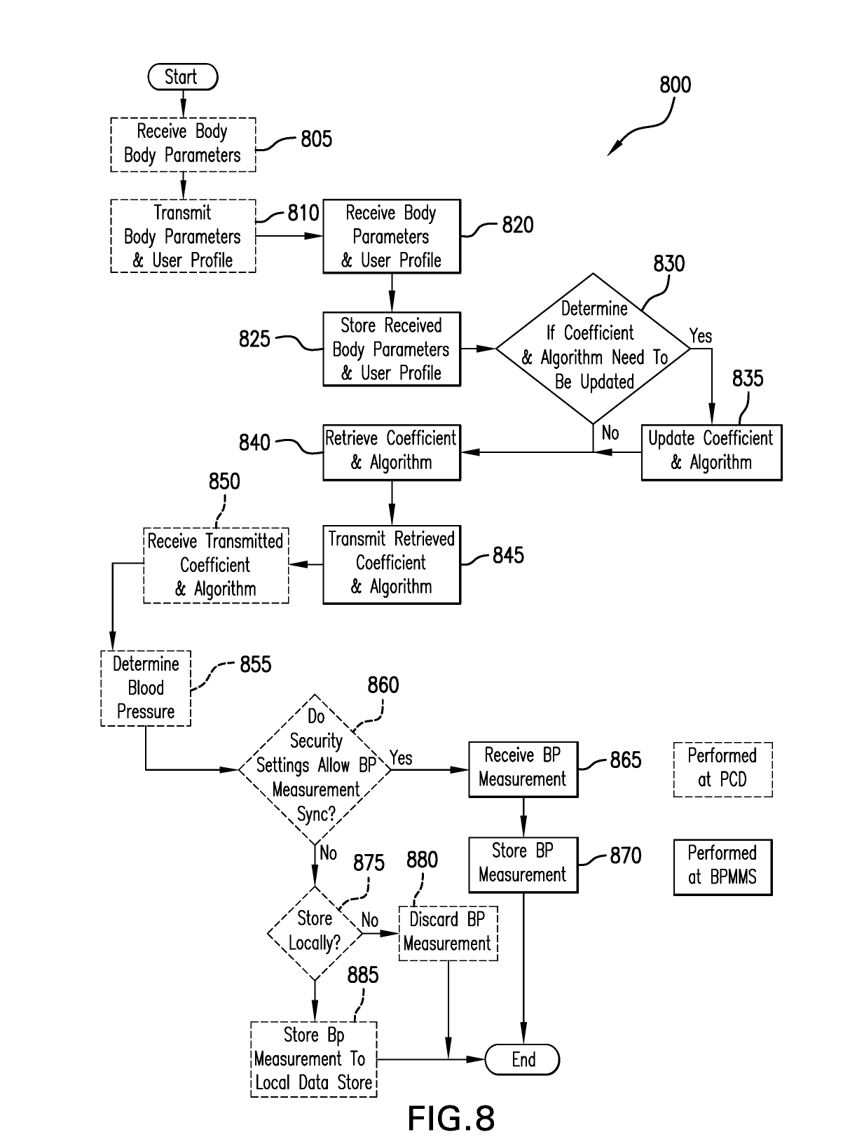

To aid understanding, this document is organized as follows. First, an exemplary use of a PCD to measure a blood pressure of a user is briefly introduced with reference to FIG. 1. Second, with reference to FIGS. 2-3, the discussion turns to exemplary embodiments that illustrate the hardware architecture and a home screen for measuring a blood pressure of a user. With reference to FIGS. 4-6, software flowcharts illustrate various operations for measuring a blood pressure with a PCD. With reference to FIG. 7, a blood pressure monitoring management system illustrates various applications associated with measuring a blood pressure with a PCD. Finally, with reference to FIG. 8, a flowchart of operations executed between a PCD and a BPMMS for updating and retrieving coefficients and algorithms tailored for an individual user.

FIG. 1 depicts an exemplary use of a PCD for measuring a blood pressure of a user. A portable communication device (PCD) 100 includes a display screen 105. The PCD 100 may be a smartphone, such as an iPhone®, commercially available from Apple of Washington, or a smartphone for Android™, commercially available from Google of California. As depicted, the display screen 105 illustrates body parameters 110 associated with a user 120. The user 120 holds the PCD 100 with a hand of a first arm 125 positioning a finger of the first arm 125 such that the finger aligns with a camera 130 of the PCD 100. The person, with the finger aligned with the camera 130, positions a microphone 135 of the PCD 100 proximate to an antecubital artery of the second arm 140 of the user 120. The PCD 100 simultaneously records a pulse flow, via the camera 130, and a pulsation, via the microphone 130. In response to the recorded pulse flow and the recorded pulsation, the PCD 100, in accordance with the inputted body parameters, determines a systolic blood pressure 140 and a diastolic blood pressure 150. The display screen 105 displays the systolic blood pressure 140 and the diastolic blood pressure 150.

In some embodiments, the body parameters 110 may include medical conditions for the user 120 administering the blood pressure measurement. For example, the body parameters 110 may include whether the user 120 suffers from diabetes. The body parameters 110 may modify an algorithm for specific use with the user 120. For example, a second person having a same gender, height and weight as the user 120, but having a different age, may require modification to the algorithm used for determining the blood pressure of the second person.

In various embodiments, the PCD 100 may store inputted and recorded information to a data store. The data store may be in the PCD 100, or in a remote database accessible from the PCD 100 via a communication network. The stored information may be used to analyze trends of the blood pressure for the user 120. In some embodiments, the stored information may be used to create trends for various body parameters, such as, for example, weight loss and weight gain. The data store may be used to create statistics to improve the accuracy of an algorithm associated with the user 120.

FIG. 2 depicts a schematic view of an exemplary PCD for measuring a blood pressure of a user. A PCD 200 includes a processor 205 operably connected to an input interface 210. The input interface 210 may be used by a user 120 to input the user's body parameters 110. The processor 205 operably connects to an input sound processor 215 to receive input sound signals from a microphone 220. The processor 205 operably connects to an image processor 225 to receive image information from a camera 230. A light source 235 in proximity to the camera 235 operably connects to the processor 205. The processor 205 operably connects to a random-access-memory module (RAM) 240 and a storage device 245. The storage device module 245 may be a data store. A network interface 250 transmits communication information between the processor 205 and an external source (not shown). A display processor 255 operably connects to the processor 205 to receive image information for display on the display screen 105.

In an illustrative embodiment, the processor 205 may transmit positioning instructions to the display processor 255 via the image processor 225 to be displayed on the display screen 105. The positioning instructions may include instructions to the user 120 for positioning the PCD 200 such that the camera 230 aligns with a finger of the first arm 125 of the user 120 and the microphone 220 of the PCD 200 may be proximate to an antecubital artery of the second arm 140 of the user 120. In some embodiments, the PCD 200 may transmit an output sound signal to a speaker (not show) to indicate proper positioning of the PCD 200. In various embodiments, the PCD 200 may transmit an output sound signal to indicate the PCD 200 is not properly positioned to begin recording a pulse flow and a pulsation of the user.

In some embodiments, the storage device 245 may include instructions for execution by the processor 205. For example, the storage device 245 may include instructions for execution by the processor 205 to initiate, via the input sound processor 215, the microphone 220 to begin recording sound samples from the antecubital artery of the user 120. In various embodiments, the storage device 245 may include instructions for execution by the processor to initiate the camera 230, via the image processor 225, and the light source 235 to begin recording image frames from the finger of the user 120 to determine pulse flow. In various embodiments, the storage device 245 may include instructions for the processor 205 to simultaneously record the sound samples and the image frames.

In some embodiments, the processor 205 may transmit to the display processor 255 status updates for display on the display screen 105. The status updates may indicate to the user 120 progress information concerning a recording of the sound samples and image frames. For example, an empty status bar may be displayed to indicate the recording of sound samples and image frames has not commenced. When the recordings begin, the status bar may fill incrementally to indicate progress. The progress indicated may be time transpired or time remaining. In various embodiments, a fixed number of sound samples and image frames may need to be recorded, for example, a sample size of thirty may be required for a more accurate blood pressure measurement. The status bar may indicate how many sound samples and image frames have been recorded or remain to be recorded. In some embodiments, multiple status bars may indicate statuses for different processes.

FIG. 3 depicts a home screen for an exemplary blood pressure measuring application running on a PCD. In the illustrated embodiment, the home screen 300 includes a pair of soft switches 305a, 305b. As depicted, the soft switches 305a, 305b may be toggled to select a specific parameter associated with the user 105. For example, the soft switch 305a may be a toggle between a “Male” position and a “Female” position to indicate the sex of the user 105. Soft switch 305b may be toggled between a “Yes” position and a “No” position to indicate whether the user 105 has diabetes. In some embodiments, more soft switches may be included on the home screen 300. Each soft switch may indicate a distinct parameter for the user 105. For example, a soft switch may be used to indicate whether a blood pressure measurement should be transmitted to an external source, such as, for example, the user's 120 doctor.

The home screen 300 includes input fields 310a-c to receive, via the input interface 210, inputted body parameters about the user 105. As depicted, the input field 310a is configured to receive the weight of the user 105. The input field 310b is configured to receive the height of the user 105 and the input field 310c is configured to receive the age of the user 105. In response to the received body parameters from the input fields 310a-c, the processor 205 may retrieve a blood pressure measurement algorithm tailored according to the received body parameters of the user 105. In some embodiments, more input fields may be included on the home screen 300 and configured to receive different information. For example, the user 105 may input the amount of sleep averaged in a 24-hour span.

The home screen 300 includes display fields 315a-j to display values determined via a blood measuring algorithm. As depicted, the display field 315a displays a determined blood speed of the user 105. The display fields 315b, 315c display a determined body surface area and a determined stroke volume, respectively, for the user 105. The display fields 315d, 315e display a determined systolic blood pressure and a determined diastolic blood pressure, respectively, for the user 105. As depicted, display fields 315f-315j display, respectively, a determined body water, a determined fat-free mass, a determined normal body mass index, a determined body mass index, and a determined pulse pressure. In some embodiments, the display fields may display other types of information. For example, a normal body mass index (NBMI) can be displayed as a reference point.

The home screen 300 includes a status display field 320. As depicted the status display field 320 is configured to display real-time data progression information. In the illustrated embodiment, thirty recordings of the pulse flow and pulsation are required to measure the blood pressure of the user 120. The status display field 320 displays the amount of recordings taken over the total amount of recordings needed. In some embodiments, the status display field 320 may display the number of recordings remaining. The home screen 300 includes a status bar 325 to indicate a progress of a specific operation. For example, the status bar 325 may be an illustrative depiction of the information displayed via status display field 320. In various embodiments, the status bar 325 may indicate progress information not associated with the information displayed via the status display field. 320.

The home screen 300 includes display fields 330, 335 to display pulse information and pulse oxygenation levels (SpO2), respectively. As depicted, a pulse graph 340 illustrates a heart rate for the user 120. The pulse graph 340 may be used to indicate whether the camera 230 is receiving blood flow information. The home screen 300 includes a sound graph 345. The sound graph 345 may indicate whether the microphone 220 is receiving pulsation information about the user 120. The home screen 300 includes a camera window 350. The camera window 350 may show whether a finger of the user 120 properly aligns with the camera 130 to indicate proper usage of the camera for determining a blood pressure of the user 120. In some embodiments, the camera window 350 may indicate whether a finger of the user 120 entirely or partially covers the camera 130.

In some embodiments, the home screen 300 may arrange the input fields, the soft switches, the display fields, and the graphs in a different manner. In various embodiments, different screens may be included to display distinct information, such as, for example, a screen may include the input fields 310a-c without the display fields or the graphs. Multiple screens may be included to guide a user 120. For example, a first screen may be included such that the user 120 needs to fill in the input fields before the user 120 may continue to a next step. A next step may be a screen displaying instruction for positions the PCD 200 for proper operations of a retrieved blood measuring algorithm.

FIG. 4 depicts a flowchart for an exemplary set of steps to measure a user's blood pressure with a PCD. The user 120 begins, at 405, by inputting, into the PCD 200, the user's 120 body parameters 110. At 410, after the PCD 200 receives the body parameters 110, the PCD 200 records pulse signals from the user 120. The PCD 200, at 415, processes the recorded signals. If the recorded signals do not exceed a predetermined threshold, the PCD 200 continues to record pulse signals, at 410. If the recorded signals do exceed a predetermined threshold, the PCD 200 determines, at 425, a systolic blood pressure for the user 120. At 430, the PCD 200 determines the diastolic blood pressure for the user 120. At 435, the PCD 200 displays, via the display screen 105, the determined systolic blood pressure, from 425, and the determined diastolic blood pressure, from 430.

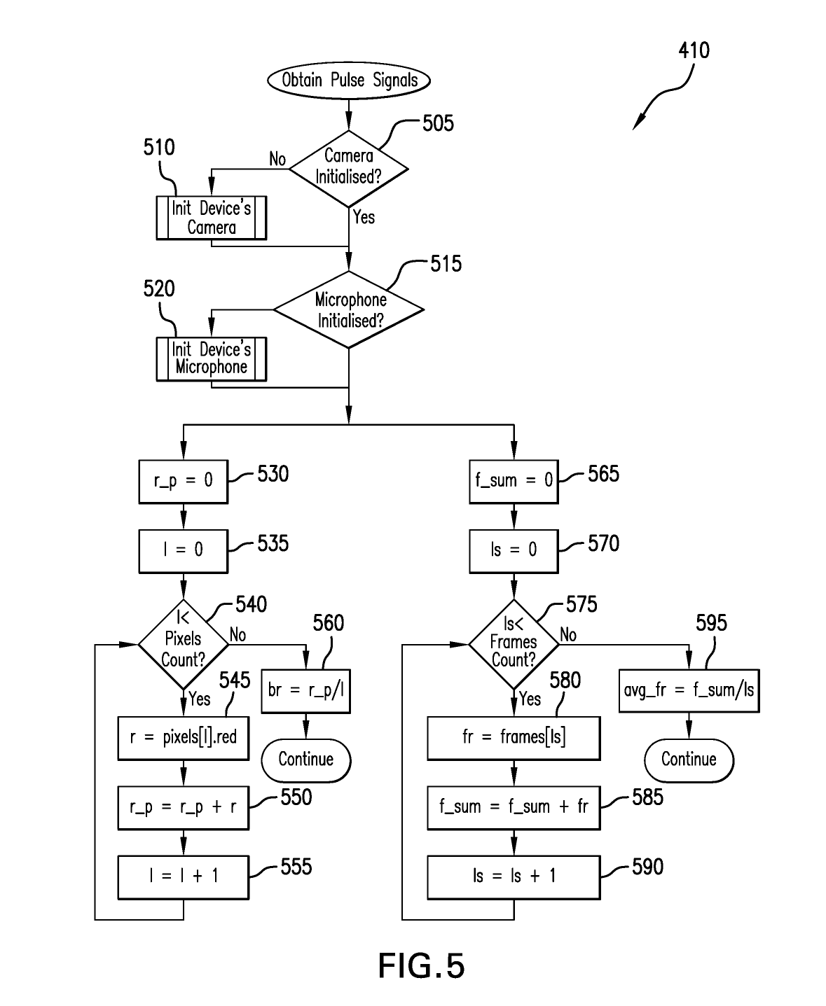

FIG. 5 depicts a flowchart of an exemplary method of using a PCD to capture image frames and sound samples. In an illustrative embodiment, the processor 205 begins execution of the method 410 by determining whether the camera 230 is initialized, at 505. If the camera 230 is not initialized, the processor 205 initializes, at 510, the camera 230. If the camera 230 is initialized, the processor 205 determines, at 515, whether the microphone 220 is initialized. If the microphone 220 is not initialized, the processor 205 initializes, at 520, the microphone 220. If the microphone 220 is initialized, the processor 205 simultaneously captures image frames from the camera 230 and captures sound samples from the microphone 220.

At 530, the processor 205 assigns a variable r_p (red plane) a value of zero. The variable r_p represents the sum of all red values of a captured frame. For example, each pixel has a red value of 0-255 and the variable r_p represents the sum of all red values in the each red pixel of a frame. At 535, the processor 205 assigns a counter variable (i) a value of zero. At 540, the processor determines whether the value of the counter variable (i) exceeds a predetermined pixel count threshold. If the value of the counter variable (i) does not exceed the predetermined pixel count threshold, a variable (r) receives, at 545, an assigned value equal to the number of red pixels for a frame at the counter variable (i). The variable (r_p) increases, at 550, by the value of the variable (r), from 545. The counter variable (i) increases by one, at 555. The processor 205 repeats, at 540, determining whether the most recent value of the counter variable (i) is less than the predetermined pixel count threshold.

If, at 540, the counter variable (i) exceeds the predetermined pixel count threshold, the processor 205 determines, at 560, a quotient blood rate such that the quotient blood rate equals the most recent value of r_p divided by the value of the most recent counter variable (i).

At 565, the processor 205 assigns a variable (f_sum) a value of zero. The variable f_sum represents sum of all values of all captured audio samples. For example, each audio sample has an audio value that is a sound level. The variable f_sum represents the sum all audio values in all audio samples. At 570, the processor 205 assigns a sample counter variable (Is) a value of zero. At 575, the processor determines whether the value of the sample counter variable (Is) exceeds a predetermined audio sample threshold. If the value of the sample counter variable (Is) does not exceed the predetermined audio sample threshold, a variable (fr) receives, at 580, an assigned value equal to the number of audio detections for an audio sample at the sample counter variable (Is). The variable (f_sum) increases, at 585, by the value of the variable (fr), from 580. The sample counter variable (Is) increases by one, at 590. The processor 205 repeats, at 575, determining whether the most recent value of the sample counter variable (Is) is less than the predetermined audio sample threshold.

If, at 575, the sample counter variable (Is) exceeds the predetermined audio sample threshold, the processor 205 determines, at 560, a quotient average audio sample (avg_fr) such that the quotient average audio sample equals the most recent value of f_sum divided by the value of the most recent sample audio counter variable (Is).

In some embodiments, the predetermined pixel count threshold may be equal to 50. The predetermined pixel count threshold and the predetermined audio sample threshold may be equal, such as, for example, both the predetermined pixel count threshold and the predetermined audio sample threshold may both equal thirty. In various embodiments, the predetermined pixel count threshold and the predetermined audio sample threshold may be different values. For example, the predetermined pixel count threshold may equal forty and the predetermined audio sample threshold may equal sixty.

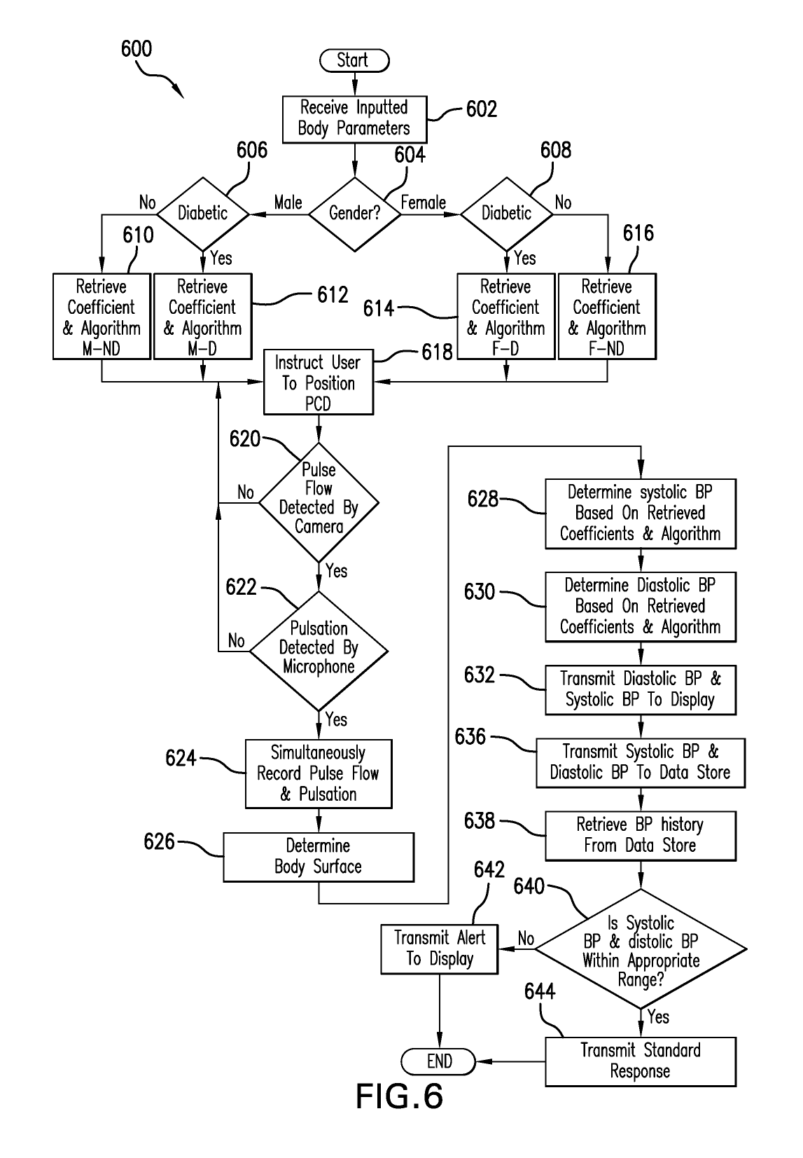

FIG. 6 a flowchart of an exemplary method of using a PCD to measure a blood pressure for a user. In some examples, some of the steps in a method 600 may be included in a program of instructions stored in the data storage device 245. The method 600 illustrates an exemplary process for determining a user's 120 blood pressure, the blood pressure being a function of the inputted body parameters 110, pulse propagation time, body surface area, body water, fat-free mass, body mass index, stroke volume, and pulse pressure. In some embodiments, a normal body mass index may be included as a parameter from which the blood pressure is a function thereof. The method 600 receives, at 602, inputted body parameters 110 via the input interface 220. The processor 205, based on the inputted body parameters 110, determines, at 604, the gender of the user 120. At 606 and 608, the processor 205 determines, based on the inputted body parameters 110, whether the user is diabetic.

At 606, if a male user is not diabetic, the processor 205 retrieves from the data store 245, at 610, a set of coefficients and a blood measuring algorithm (M-ND) based on the inputted body parameters 110. If a male user is diabetic, the processor 205 retrieves from the data store 245, at 612, a set of coefficients and a blood measuring algorithm (M-D) based on the inputted body parameters 110. At 608, if a female user is diabetic, the processor 205 retrieves from the data store 245, at 614, a set of coefficients and a blood measuring algorithm (F-D) based on the inputted body parameters 110. If a female user is not diabetic, the processor 205 retrieves from the data store 245, at 616, a set of coefficients and a blood measuring algorithm (F-ND) based on the inputted body parameters 110. In various embodiments, the data store may be at a remote location. The processor 205 may transmit and retrieve information from the remote data store via the network interface 250.

At 618, the processor transmits, via the display processor 255, to the display 105 instructions for positioning the PCD 200 such that a finger of a first arm 125 aligns with the camera 230 and positions a microphone 220 proximate to an antecubital artery of the second arm 140 of the user 120. At 620, the processor determines whether a pulse flow is detected by the camera 230. If a pulse flow is not detected, the processor re-transmits, at 618, positioning instructions to the display 105. If a pulse flow is detected, the processor 205 determines whether a pulsation is detected by the microphone 220, at 622. If a pulsation is not detected, the processor re-transmits, at 622, positioning instructions to the display 105.

If a pulsation is detected, at 622, the processor 205 simultaneously records, at 624, received information from the camera 230 and the microphone 220. The received information may be captured via the method 410. The processor determines, at 626, a body surface for the user 105 based on the retrieved coefficients and body measuring algorithm in accordance with the inputted body parameters 110. For example, if the inputted body parameters indicate the user is a female with diabetes, the processor may user the coefficients and body measuring algorithm (F-D), from 614, to determine the body surface of the user.

At 628, the processor 205, in response to the record information from the camera 230 and the microphone 220, determines an average time difference between the pulse flow and the pulsation to determine a pulse propagation time. The processor 205 determines a body mass index and a normal body mass index. The processor 205 determines a body mass index correlation using the determined body mass index and the determined normal body mass index. Using a predetermined coefficient (k) retrieved from the data store 245 and the determined body mass index correlation, the processor 205 determines a systolic blood pressure. In some embodiments, the predetermined coefficient (k) is determined through statistical research based on recorded pulse propagation and blood pressure measurements recorded using a vital signs monitor, such as, for example, a Spot Vital Signs® Device as distributed by WelchAllyn®, Inc. of New York. At 630, the processor 205 determines a body surface area, a body water, a fat-free mass, a stroke volume, and a pulse pressure. The processor 205 determines a diastolic blood pressure. In some embodiments, the diastolic pressure may be the difference between the predetermined systolic pressure and the pulse pressure. At 632, the processor 205 transmits the determined systolic blood pressure and the determined diastolic blood pressure to the display 105 via the display processor 255.

The processor 205, at 636, transmits the determined systolic blood pressure and the determined diastolic blood pressure to the data store 245 to store. At 638, the processor 205 retrieves, from the data store 245, a blood pressure history for the user 120. In some embodiments, the processor 205 may transmit the inputted body parameters, the determined systolic blood pressure and the determined diastolic blood pressure to a remote data store via the network interface 250. The processor 205 may receive, from the remote data store via the network interface, a blood pressure history for the user 120. In various embodiments, the processor 205 may retrieve from the remote data store the coefficients and body measuring algorithm for the user 105 based on the inputted parameters.

At 640, it is determined whether the determined systolic blood pressure and the determined diastolic blood pressure are within a predetermined blood pressure range. The predetermined blood pressure range may be based on the inputted body parameters 110 of the user 120. If the determined systolic blood pressure and the determined diastolic blood pressure are not within the predetermined blood pressure range, the processor 205 transmits, at 642, an alert to the display 105 via the display processor 255. The alert may advise the user 120 to measure the user's 120 blood pressure again. If the determined systolic blood pressure and the determined diastolic blood pressure are within the predetermined blood pressure range, the processor 205 may transmit, at 644, a message indicating a successful measurement of blood pressure to the display 105.

In some embodiments, the predetermined blood pressure range may be based on a combination of user's 105 blood pressure history and the inputted body parameters. In various embodiments, the processor 205 may transmit, via the network interface 250, the determined systolic blood pressure and the determined diastolic blood pressure to a remote medical facility. For example, the determined information may be transmitted to a doctor's office for analysis or to assist in a diagnosis of the user 120. In various embodiments, if the determined systolic blood pressure and the determined diastolic blood pressure are not within the predetermined blood pressure range based on the inputted body parameters, the processor 205 may execute a self-diagnosis routine to ensure proper functioning of the PCD 200.

FIG. 7 depicts an illustration of an exemplary blood pressure monitoring management system (BPMMS) integrated for use multi-use applications. A user 605 measures the user's 705 blood pressure 715 using a PCD 710. The PCD 710 transmits, via a network interface 720, the measured blood pressure 715 to a blood pressure monitoring management system (BPMMS) 725. The BPMMS 725 includes a user profile engine 730. The user profile engine 730 may associate a unique user identification (UID) with information received about the user's 705 measured blood pressure 715. A data store, also included in the BPMMS 725, may store the received information such that the information may be retrieved using the user's UID. In some embodiments, other information, such as, for example, the user's 705 body parameters, may be transmitted from the PCD 710 and received by the user profile engine 730 to configure for storing.

A coefficient and algorithm engine (CAE) 740, also included in the BPMMS 725, may determine a set of coefficients and a blood pressure algorithm based on the stored information, in the data store 735 associated with the UID of the user 705. The CAE 740 may transmit the determined set of coefficients and blood pressure algorithm to the PCD 710 for use in measuring a blood pressure of the user 705. In some embodiments, as information continues to be stored in the data store 735, the CAE 740 may continuously modify the set of coefficients and the blood pressure algorithm associated with the UID of the user 705 such the accuracy of the blood pressure measurements for the user 705 increases as more information about the user 705 is stored in the data store 735.

The BPMMS 725 includes a recommendation engine 745. The recommendation engine 745 may transmit, in response to a user's 705 blood pressure measurement 715, recommendations from the BPMMS 725 to the PCD 710. For example, if the blood pressure measurement 715 indicates that the user is experiencing high blood pressure, the recommendation engine 715 may transmit a recommendation to the user 705 that an appointment be scheduled with a doctor. In some embodiments, the recommendation engine may propose appointment times based a user's 705 doctor's office calendar, and in response to the user 705 selecting an appointment time, schedule the appointment with the user's 705 office.

The BPMMS 725 further includes a third party engine 750. The third party engine 705 may be configured to communicate with a third party fitness application 755. The third party fitness application includes a user profile engine 760, an activity engine 765, an analytics engine 770, and data store 775. The third party engine 750 may receive, via a network interface 780, from the user profile engine 760 identifying information about the user 705 such that the user profile engine 730 associates the received identifying information with the UID of the user 705. In response to associating the received identifying information with the UID, the third party engine 750 may receive from the activity engine 765 and the analytics engine 770 activity data (e.g., types of activities such as running) and activity trends data (e.g., amount of daily activity time). The received activity data and the activity trends data may be stored in the data store 735. An analytic engine 785 of the BPMMS 725, may perform analytics on the stored data in the data store 735. For example, the analytic engine may determine trends about the user's 705 blood pressure 715 as it relates to the user's activity data. The results of the analytics performed by the analytic engine 735 may be transmitted to the PCD 710 for display or to a doctor to assist in a diagnosis of the user 705.

A doctor 785 may retrieve the user's 705 blood pressure measurement 715 in real-time. For example, a doctor may conduct a consultation with the user 705 over a telephone. During the consultation, the doctor 785 may request the user 705 perform a blood pressure measurement 715 to assist the doctor 785 with a diagnosis. In response to the user 705 using the PCD 710 to take a blood pressure measurement 715, the blood pressure measurement may be transmitted, via the network interface 720, to a PCD 790 of the doctor 785. The PCD 790 may be a computer, an iPad©, or a tablet for use Android™. In some embodiments, the doctor 785 may retrieve from the BPMMS 725 information pertaining to the UID of the user 705. As depicted, the retrieved information may include blood pressure history, body parameters, analytics, and activity levels for the user 705. In various embodiments, the doctor 785, via the BPMMS 725, may create a schedule to define times when the user 705 needs to take a blood pressure measurement.

The BPMMS 725 includes an analytics engine 795. The analytics engine 795 may generate trends based on the information stored in the data store 735. For example, the analytics engine may generate a graph charting the relationship between the user's 120 weight and the user's 120 blood pressure. In various embodiments, the analytics engine 795 may factor in information received from a third party app. For example, the analytics engine may generate a graph illustrating the user's 120 blood pressure as it relates to the user's 120 activity levels. The generated trends may be transmitted to the PCD 710 to be displayed for the user 120 to review. In some embodiments, the generated trends may be transmitted to the PCD 790 for the doctor 785 to review.

FIG. 8 depicts a flowchart of an exemplary PCD-BPMMS operations executed between a PCD and a BPMMS. In some examples, some of the steps in a method 800 may be included in a program of instructions. The program of instructions may be stored in either the data storage device 245 or the BPMMS 725. In some embodiments, the PCD 200 may store a subset of the program of instructions in the data store device 245, and the BPMMS 725 may store a remaining subset of the program of instructions in the data store 735.

At 805, the PCD 200 receives, via the input interface 210, inputted body parameters. The PCD 200 associates the inputted body parameters with a previously create user profile. The user profile may include identifying information, such as, for example, a name and a birthday, to identify the user 120. In some embodiments, the PCD 200 may include a multitude of user profiles such that each profile is associated with an individual user. An individual user may select a user profile that corresponds to the individual user before inputting the body parameters.

At 810, the PCD 200 transmits, via the network interface 250, the inputted body parameters with the associated user profile to the BPMMS 725. At 820, the BPMMS 725 receives, via the network interface 780, the transmitted inputted body parameters and the associated user profile. At 825, the BPMMS 725 stores the received inputted body parameters such that the received user profile may be used to retrieved the saved inputted body parameters.

At 830, the BPMMS 725 determines whether a set of coefficients and an algorithm associated with the received user profile needs to be modified in response to the received inputted body parameters. For example, if a weight of the user 120 increases since a previous measurement, the set of coefficients and an algorithm associated with the user 120 may need to be modified to account for the increase in weight. In some embodiments, the determination of whether the set of coefficients and the algorithm associated with the received user profile needs to be modified may include factors received from a third party. For example, the BPMMS 725 may receive, from the fitness app 755, data about the activity levels of the user 120. In the event the user's 120 activity levels have decreased, the CAE 740 may need to modify the set of coefficients and the algorithm to reflect the decrease in activity levels.

If the BPMMS 725 determines that the set of coefficients and the algorithm associated with the received user profile needs to be modified, the BPMMS 725, via the CAE 740, modifies, at 835, the set of coefficients and the algorithm associated with the received user profile to reflect the received inputted body parameters. In various embodiments, the accuracy of the of the blood pressure measurement may increase as the set of coefficients and the algorithm are modified over time. If the BPMMS 725 determines that the set of coefficients and the algorithm associated with the received user profile does not need to be modified, the set of coefficients and the algorithm associated with the received user profile remain unchanged. In some embodiments, a user profile may not have an associated set of coefficients nor an associated algorithm. As such, the BPMMS 725 may provide a default set of coefficients and a default algorithm based only on the user inputted body parameters.

At 840, the BPMMS 725 retrieves the set of coefficients and the algorithm associated with the received user profile as determined from 830. The BPMMS 725 transmits, at 845, the retrieved set of coefficients and the retrieved algorithm to the PCD 200. The PCD 200 receives, at 850, the transmitted set of coefficients and the transmitted algorithm. At 855, the PCD 200 uses the set of coefficients and the algorithm received, from 850, along with blood flow and pulsation information received from the camera 230 and the microphone 220 to determine a blood pressure measurement for the user 120.

At 860, the PCD 200 determines whether a security setting for the associated user profile permits the syncing the determined blood pressure measurement with the BPMMS 725. If the security setting does permit syncing, the PCD 200 transmits the determined blood pressure measurement to the BPMMS 725. At 865, the BPMMS 725 receives the transmitted blood pressure measurement. The BPMMS 725 stores, at 870, the received blood pressure measurement. In some embodiments, the BPMMS 725 may store the blood pressure measurements such that a third party (e.g., doctor's office) may retrieve the blood pressure measurements associated with an individual user profile based on security setting of the BPMMS 725.

If the security setting does not permit syncing, the PCD 200 determines, at 875, whether to store the blood pressure measurement locally. If the blood pressure measurement is not to be stored locally, the PCD 200 discards, at 880, the blood pressure measurement. If the blood pressure measurement is to be stored locally, the PCD 200 stores, at 885, the blood pressure measurement to the data store device 245. In various embodiments, a user may manually sync blood pressure measurements at a later time. For example, the user may decide to store a number of blood pressure measurements locally and sync the stored blood pressure measurements at a same time. In some embodiments, the BPMMS 725, via the analytics engine 795, may generate trends to be used with inputted body parameters in determining a set of coefficients and an algorithm.

Although various embodiments have been described with reference to the Figures, other embodiments are possible. For example, the blood pressure measurement may be directed towards a mobile software application (App) that resides on a mobile device, such as a smartphone. The App may measure a user's blood pressure without the need for any external devices, such as, for example, an arm cuff. When the App is activated to measure blood pressure, the App, through the camera and the microphone of a mobile device, may gather pulse data from a user's index finger and elbow artery. In response to the gathered pulse data, the App may use the user's input body parameters (e.g., gender, age, weight, height) to calculate systolic and diastolic pressure and may display the results on a graphical display of the mobile device.

In some embodiments the BPMMS 725 may include a HIPPA management engine to manage hippa compliance of the information being received, stored, and transmitted. In various embodiments, the blood pressure measurement system may include a reminder engine to notify a user of a next scheduled blood pressure measurement. In some embodiments, the reminder engine may alert a user after a predetermined time period has been exceed since the most recent blood pressure measurement. In various embodiments, the reminder engine may be included in the BPMMS 725.

Some aspects of embodiments may be implemented as a computer system. For example, various implementations may include digital and/or analog circuitry, computer hardware, firmware, software, or combinations thereof. Apparatus elements can be implemented in a computer program product tangibly embodied in an information carrier, e.g., in a machine-readable storage device, for execution by a programmable processor; and methods can be performed by a programmable processor executing a program of instructions to perform functions of various embodiments by operating on input data and generating an output. Some embodiments can be implemented advantageously in one or more computer programs that are executable on a programmable system including at least one programmable processor coupled to receive data and instructions from, and to transmit data and instructions to, a data storage system, at least one input device, and/or at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example and not limitation, both general and special purpose microprocessors, which may include a single processor or one of multiple processors of any kind of computer. Generally, a processor will receive instructions and data from a read-only memory or a random access memory or both. The essential elements of a computer are a processor for executing instructions and one or more memories for storing instructions and data. Storage devices suitable for tangibly embodying computer program instructions and data include all forms of non-volatile memory, including, by way of example, semiconductor memory devices, such as EPROM, EEPROM, and flash memory devices; magnetic disks, such as internal hard disks and removable disks; magneto-optical disks; and, CD-ROM and DVD-ROM disks. The processor and the memory can be supplemented by, or incorporated in, ASICs (application-specific integrated circuits). In some embodiments, the processor and the member can be supplemented by, or incorporated in hardware programmable devices, such as FPGAs, for example.

In some implementations, each system may be programmed with the same or similar information and/or initialized with substantially identical information stored in volatile and/or non-volatile memory. For example, one data interface may be configured to perform auto configuration, auto download, and/or auto update functions when coupled to an appropriate host device, such as a desktop computer or a server.

In some implementations, one or more user-interface features may be custom configured to perform specific functions. An exemplary embodiment may be implemented in a computer system that includes a graphical user interface and/or an Internet browser. To provide for interaction with a user, some implementations may be implemented on a computer having a display device, such as an LCD (liquid crystal display) monitor for displaying information to the user, a keyboard, and a pointing device, such as a mouse or a trackball by which the user can provide input to the computer.

In various implementations, the system may communicate using suitable communication methods, equipment, and techniques. For example, the system may communicate with compatible devices (e.g., devices capable of transferring data to and/or from the system) using point-to-point communication in which a message is transported directly from the source to the first receiver over a dedicated physical link (e.g., fiber optic link, point-to-point wiring, daisy-chain). The components of the system may exchange information by any form or medium of analog or digital data communication, including packet-based messages on a communication network. Examples of communication networks include, e.g., a LAN (local area network), a WAN (wide area network), MAN (metropolitan area network), wireless and/or optical networks, and the computers and networks forming the Internet. Other implementations may transport messages by broadcasting to all or substantially all devices that are coupled together by a communication network, for example, by using Omni-directional radio frequency (RF) signals. Still other implementations may transport messages characterized by high directivity, such as RF signals transmitted using directional (i.e., narrow beam) antennas or infrared signals that may optionally be used with focusing optics. Still other implementations are possible using appropriate interfaces and protocols such as, by way of example and not intended to be limiting, USB 2.0, Fire wire, ATA/IDE, RS-232, RS-422, RS-485, 802.11a/b/g, Wi-Fi, Ethernet, IrDA, FDDI (fiber distributed data interface), token-ring networks, or multiplexing techniques based on frequency, time, or code division. Some implementations may optionally incorporate features such as error checking and correction (ECC) for data integrity, or security measures, such as encryption (e.g., WEP) and password protection.

A number of implementations have been described. Nevertheless, it will be understood that various modification may be made. For example, advantageous results may be achieved if the steps of the disclosed techniques were performed in a different sequence, or if components of the disclosed systems were combined in a different manner, or if the components were supplemented with other components. Accordingly, other implementations are contemplated.

What is claimed is:

1. A method of measuring a human's blood pressure, the method comprising:
receiving, in a portable communication device (PCD), user input information about body parameters of an operator;
simultaneously recording:
a blood flow information by positioning a camera of the PCD such that the camera aligns with a finger of a first arm;
a pulsation information by positioning a microphone of the PCD such that the microphone proximate to an antecubital artery of a second arm;
determining, in response to the recorded blood flow information and the recorded pulsation information, a pulse velocity;
determining, in response to the determined pulse velocity, a systolic blood pressure measurement;
determining, in response to the inputted body parameters and the recorded pulsation information, a pulse pressure;
determining, in response to the determined pulse pressure, a diastolic blood pressure measurement; and,
transmitting, the determined systolic blood pressure and the determined diastolic blood pressure for display on a display panel of the PCD.

2. The method of claim 1, further comprising associating a unique user profile to the received inputted body parameters and the determined systolic blood pressure and the determined diastolic blood pressure.

3. The method of claim 2, further comprising storing the received inputted body parameters, the determined systolic blood pressure, and the determined diastolic blood pressure in a data store such that the stored inputted body parameters, the stored systolic blood pressure, and the stored diastolic blood pressure is configured to be retrieved via the unique user profile.

4. The method of claim 3, wherein the data store is configured to share the stored information with a remote PCD.

5. The method of claim 2, further comprising retrieving, from the data store, a set of coefficients and a blood measuring algorithm, wherein the retrieved set of coefficients and the retrieved blood measuring algorithm are determined based on the stored inputted body parameter, the stored systolic blood pressure measurements and the stored diastolic blood pressure measurements.

6. The method of claim 5, wherein the data store comprises a remote location.

7. The method of claim 6, further comprising transmitting, via a network interface, the unique user profile, the stored inputted body parameter, the stored systolic blood pressure measurements and the stored diastolic blood pressure measurements to be stored in the data store.

8. The method of claim 2, further comprising analyzing the stored inputted body parameter, the stored systolic blood pressure measurements and the stored diastolic blood pressure measurements to create a trends analysis for the unique user profile.

9. The method of claim 1, further comprising transmitting to a graphical display of the PCD positioning instructions to position the camera such that the camera aligns with a finger of the first arm, and the microphone such that the microphone proximate to an antecubital artery of the second arm.

10. The method of claim 2, further comprising receiving, from a third party, information about fitness activities associated the unique user profile.

11. A computer program product (CPP) tangibly embodied in a data store, the CPP including instructions that, when executed, operate a portable communication device (PCD) to measure the blood pressure of a user, the operations comprising:
receive, in a portable communication device (PCD), user input information about body parameters of an operator;
simultaneously record:
a blood flow information by positioning a camera of the PCD such that the camera aligns with a finger of a first arm;
a pulsation information by positioning a microphone of the PCD such that the microphone proximate to an antecubital artery of a second arm;
determine, in response to the recorded blood flow information and the recorded pulsation information, a pulse velocity;
determine, in response to the determined pulse velocity, a systolic blood pressure measurement;
determine, in response to the inputted body parameters and the recorded pulsation information, a pulse pressure;
determine, in response to the determined pulse pressure, a diastolic blood pressure measurement; and,
transmit, the determined systolic blood pressure and the determined diastolic blood pressure for display on a display panel of the PCD.

12. The CPP of claim 11, further comprising associate a unique user profile to the received inputted body parameters and the determined systolic blood pressure and the determined diastolic blood pressure.

13. The CPP of claim 12, further comprising store the received inputted body parameters, the determined systolic blood pressure, and the determined diastolic blood pressure in a data store such that the stored inputted body parameters, the stored systolic blood pressure, and the stored diastolic blood pressure is configured to be retrieved via the unique user profile.

14. The CPP of claim 12, further comprising retrieve, from the data store, a set of coefficients and a blood measuring algorithm, wherein the retrieved set of coefficients and the retrieved blood measuring algorithm are determined based on the stored inputted body parameter, the stored systolic blood pressure measurements and the stored diastolic blood pressure measurements.

15. The CPP of claim 14, wherein the data store is disposed at a remote location.

16. The CPP of claim 15, wherein the data store is configured to share the stored information with a remote PCD.

17. The CPP of claim 16, further comprising transmit, in response to a received third party request, the received inputted body parameters, the determined systolic blood pressure, and the determined diastolic blood pressure to a remote PCD.

18. The CPP of claim 12, further comprising analyze the stored inputted body parameter, the stored systolic blood pressure measurements and the stored diastolic blood pressure measurements to create a trends analysis for the unique user profile.

19. The CPP of claim 11, further comprising transmit to a graphical display of the PCD positioning instructions to position the camera such that the camera aligns with a finger of the first arm, and the microphone such that the microphone proximate to an antecubital artery of the second arm.

20. The CPP of claim 11, further comprising maintain different unique user profiles such that each unique user profile is associated with a distinct combination of the inputted body parameters.
